(19) United States
(12) Patent Application Publication
(10) Pub. No.: US 2020/0129679 A1
(43) Pub. Date: Apr. 30, 2020

(54) Dual Therapy Device and Related Method

(71) Applicants: Thomas Zink, San Antonio, TX (US); Frank Kuwamura, III, San Antonio, TX (US)

(72) Inventors: Thomas Zink, San Antonio, TX (US); Frank Kuwamura, III, San Antonio, TX (US)

(21) Appl. No.: 16/659,607

(22) Filed: Oct. 22, 2019

Related U.S. Application Data
(60) Provisional application No. 62/748,592, filed on Oct. 22, 2018.

(51) Int. Cl. A61M 1/00; A61M 25/00; A61K 35/545; A61K 35/19

(52) U.S. Cl. CPC A61M 1/0058; A61M 25/007; A61M 2210/1003; A61K 35/19; A61M 2205/0266; A61K 35/545

(57) ABSTRACT

A dual therapy device for delivering reduced pressure therapy and regenerative therapy to a degenerated vertebral disc and a method of using the same is disclosed herein. The dual therapy device is preferably comprised of a cannulated trocar for accessing a disc space, a probe that coils within said disc space and a cannulated and fenestrated catheter that also coils within the disc space, adjacent to the probe. The catheter is useful for applying reduced or negative pressure therapy to the entire or substantially entire disc space and, upon completion of said therapy, delivering regenerative therapy to the disc space in the form of a biological material. Nonetheless, it is also contemplated that the catheter may be eliminated, and the dual therapies delivered via a cannulated and fenestrated probe.

### CROSS REFERENCE

This application claims priority from U.S. Provisional Patent Application Ser. No. 62/748,592 filed on Oct. 22, 2018, which is incorporated herein by reference.

### FIELD OF THE INVENTION

The present invention relates to a dual therapy device for delivering reduced pressure therapy and regenerative therapy to a degenerated vertebral disc.

### BACKGROUND

Degenerative spine disease is a general term that refers to any disease of the spinal column that results from the aging process and/or wear and tear that occurs to the bone and soft tissues of the spine. Common types of degenerative spine disease include, without limitation, spinal osteoarthritis, degenerative disc disease, bulging discs, herniated discs, spondylolisthesis, degenerative scoliosis, bone spurs, spinal stenosis, foraminal stenosis, pinched nerves, sciatica and the like, each of which results in pain and/or discomfort for the individual with the disease.

Heretofore, mild cases of degenerative spine disease are oftentimes treated with physical therapy, hot or cold compresses, acupuncture, and/or over the counter medication, while more severe or advanced cases may require some form of surgery (e.g., laminectomies, spinal fusion, etc.) or the injection of anti-inflammatory steroid, cortisone or the like. Unfortunately, these types of treatments are not always effective, and may not provide the patient with sufficient relief or a positive outcome.

Additionally, reduced pressure and negative therapy devices are known in the art for assisting with the healing of an open wound. Such devices typically comprise both a sealant layer and a suction apparatus. The sealant layer is used to create a sealed enclosure between the device and the surface of a patient by forming, preferably, an airtight seal around an area of wounded tissue that requires negative or reduced pressure therapy. The suction apparatus is typically in communication with the sealant layer, and functions so as to reduce the amount of pressure present underneath the sealant layer. Together, the combination of the sealant layer and the suction apparatus create a closed reduced pressure therapy system. By introducing reduced pressure to a wound, circulation is improved and cellular waste is removed, both of which promote healing.

Additionally, it has been determined that applying negative or reduced pressure to bone that has been subjected to a traumatic event promotes bone healing, as well as bone growth. Current methods of applying negative or reduced pressure to an affected bone, such as a vertebra, involve placing a manifold with a tube in communication therewith in the surgical site and then closing the patient with the opposite end of the tube extending outside of the patient's body. The portion of the tube that extends outside the patient's body may then be connected to a pump that is used to remove or reduce pressure from the surgical site to start a cascade of healing on the affected bone. The cascade accelerates the healing of the bone, promotes bone growth, and, when performed on the spine, the fusion of vertebra.

However, said prior art negative or reduced pressure therapies are not without their limitations. For example, post-operatively, the manifold can become dislodged from the surgical site, for example, due to movement of the patient. If this happens, not only is the manifold dislodged within the patient, but negative pressure can no longer be applied to the bone and the benefits of applying negative pressure to increase bone healing and promote bone growth are lost.

Additionally, it is difficult to determine if the manifold has become dislodged from the surgical site unless the patient is x-rayed or re-opened, neither of which is ideal for a post-operative patient. For example, additional x-rays to locate the manifold expose the patient to unnecessary radiation, which is both undesirable and potentially dangerous, and not inexpensive. Also, additional surgery exposes the patient to undesirable risks such as those associated with anesthesia, the risk of infection, not to mention pain, discomfort, missed work and the expense of the additional surgical procedure to remove or reattach the manifold.

Consequently, there is a long felt need in the art for a reduced or negative pressure delivery device that is capable of delivering reduced or negative pressure therapy to a degenerated vertebral device. Further, there is also a long felt need in the art for a delivery device that is also capable of delivering stem cells and regenerative therapy to a degenerated vertebral device. Additionally, there is a long felt need for a dual therapy delivery device that overcomes the limitations of the prior art, and that is safe and easy to use.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The present invention discloses a dual therapy device that is capable of applying reduced or negative pressure therapy, as well as regenerative therapy (e.g., through the injection of stem cells, platelet-rich plasma, etc.), to a portion of the body such as a degenerated or collapsed spinal disc. In one embodiment, the device of the present invention is preferably comprised of a cannulated trocar, a probe or delivery inserter, a cannulated and fenestrated catheter and a pump. More specifically, and by way of example, once a patient is sedated, the dual therapy device of the present invention may be placed into the disc space under fluoroscopy. Upon insertion, the probe will coil around inside the nucleus of the degenerated or collapsed spinal disc. Once the coil is in place, the cannulated and fenestrated catheter is permitted to follow the path of the coiled probe into the disc space via the trocar. The catheter is preferably in fluid communication with a length of tube that extends outside the patient's body and is connected to, and in fluid communication with, a pump or other vacuum source. The pump, in turn, is used to introduce reduced or negative pressure to the degenerated or collapsed disc space via the catheter and the fenestrations therein. Once the reduced or negative pressure treatment is complete, the catheter may be slowly withdrawn from the disc space. As the catheter is being withdrawn, platelet-rich plasma (PRP), stem cells or other forms of regenerative biologics could be delivered via the catheter into the void left in the disc space to facilitate healing therein and bone growth.

In an alternative embodiment, the cannulated and fenestrated catheter could be eliminated so that the dual therapy device would simply be comprised of a cannulated trocar, a cannulated and fenestrated probe or other delivery inserter, and a pump. More specifically, and by way of example, once the patient is sedated, the dual therapy device of the present invention may be placed into the disc space under fluoroscopy. Upon insertion, the cannulated and fenestrated probe will coil around inside the nucleus of the degenerated or collapsed spinal disc. The probe would be in fluid communication with a length of tube that extends outside the patient's body and is connected to, and in fluid communication with, a pump or other vacuum source. The pump, in turn, is used to introduce reduced or negative pressure to the degenerated or collapsed disc space via the probe and the fenestrations therein. Once the reduced or negative pressure treatment is complete, the probe may be slowly withdrawn from the disc space. As the probe is being withdrawn, PRP, stem cells or other forms of regenerative biologics could be delivered, via the fenestrations in the probe, into the void left in the disc space to facilitate healing therein and bone growth.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

While the specification concludes with claims particularly pointing out and distinctly claiming the present invention, it is believed that the present invention will be better understood from the following description in conjunction with the accompanying FIGS., in which like reference numerals identify like elements, and wherein:

FIG. 1 illustrates a perspective view of the dual therapy device of the present invention being inserted into a disc space in accordance with the disclosed architecture.

FIG. 2 illustrates a perspective view of the delivery inserter of the dual therapy device of the present invention inserted into and beginning to coil about the interior of a disc space in accordance with the disclosed architecture.

FIG. 3 illustrates a perspective view of the delivery inserter of the dual therapy device of the present invention inserted into and continuing to coil further about the interior of the disc space in accordance with the disclosed architecture.

FIG. 4 illustrates a perspective view of the delivery inserter of the dual therapy device of the present invention inserted into and continuing to coil even further about the interior of the disc space in accordance with the disclosed architecture.

FIG. 5 illustrates a perspective view of an alternative embodiment of the delivery inserter of the dual therapy device of the present invention inserted into and coiling about the interior of the disc space in accordance with the disclosed architecture.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof.

Generally stated, the present invention relates to a combination reduced or negative pressure/regenerative therapy delivery device that is capable of both applying reduced or negative pressure therapy to an affected portion of a patient's body, such as a degenerative or collapsed spinal disc, and delivering platelet-rich plasma (PRP), stem cells or other forms of regenerative biologics to the disc space to facilitate healing and bone growth. The device may be used in an outpatient setting and the therapies provided thereby would not promote the degeneration of adjacent spinal discs, which is a somewhat common (and undesirable) outcome associated with alternative treatments such as spinal fusion surgery.

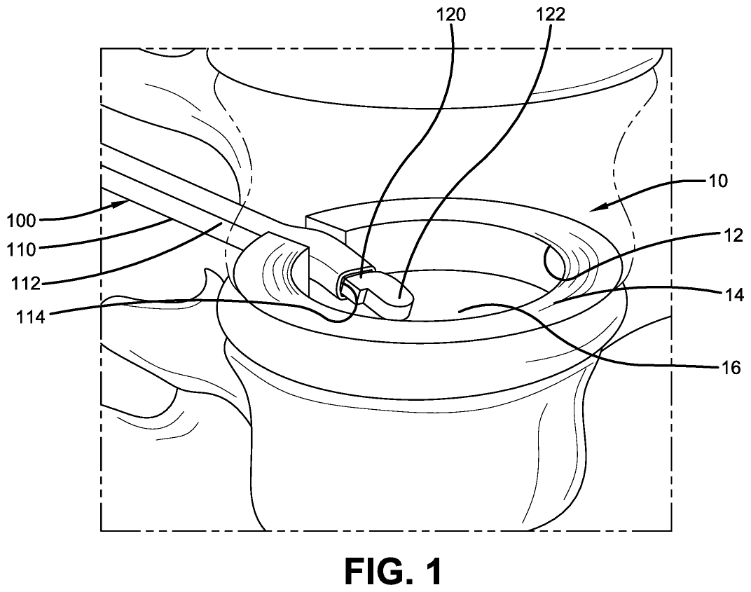

Referring initially to the drawings, FIG. 1 illustrates a perspective view of a portion of a spine 10 comprised of a disc space 12 formed by a disc annulus 14 and containing a nucleus pulposus 16. FIG. 1 also illustrates a perspective view of the dual therapy device 100 of the present invention being inserted through the disc annulus 14 and into the disc space 12 and nucleus pulposus 16.

The dual therapy device 100 is preferably comprised of a cannulated insertion device or trocar 110, a probe or delivery inserter 120, a catheter 130, and a pump 140. More specifically, the trocar 110 provides access to the disc space 12 to the surgeon or other medical professional and is preferably comprised of an elongated body portion 112 and a continuous longitudinal opening 114 therein. As explained more fully below, during a dual therapy procedure, the trocar 110 is inserted through the disc annulus 14, and into the disc space 12. Trocar 110 may be any trocar device commonly known in the art and is preferably comprised of stainless steel or medical grade plastic, but any other suitable medical grade material can also be used without affecting the overall concept of the present invention.

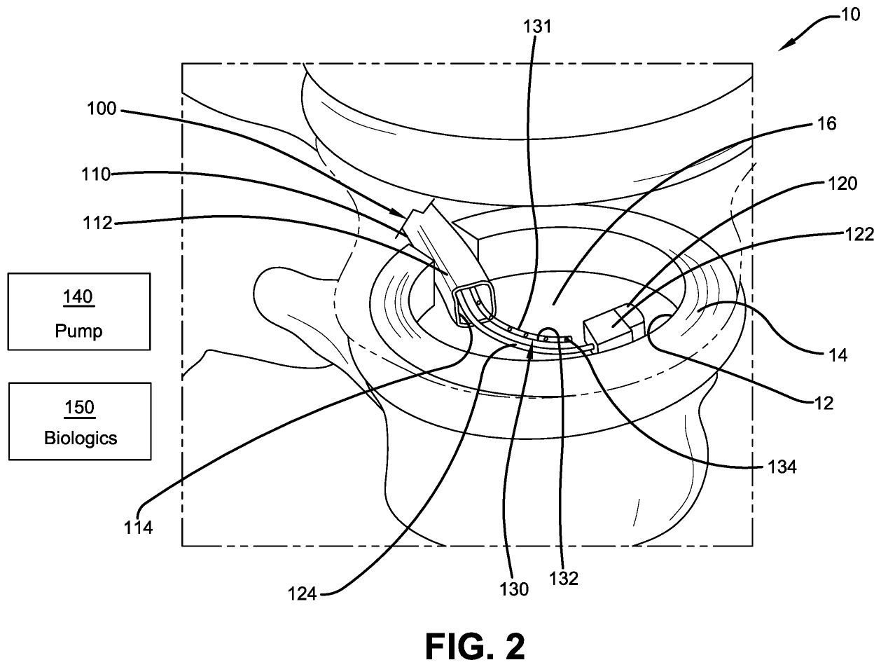

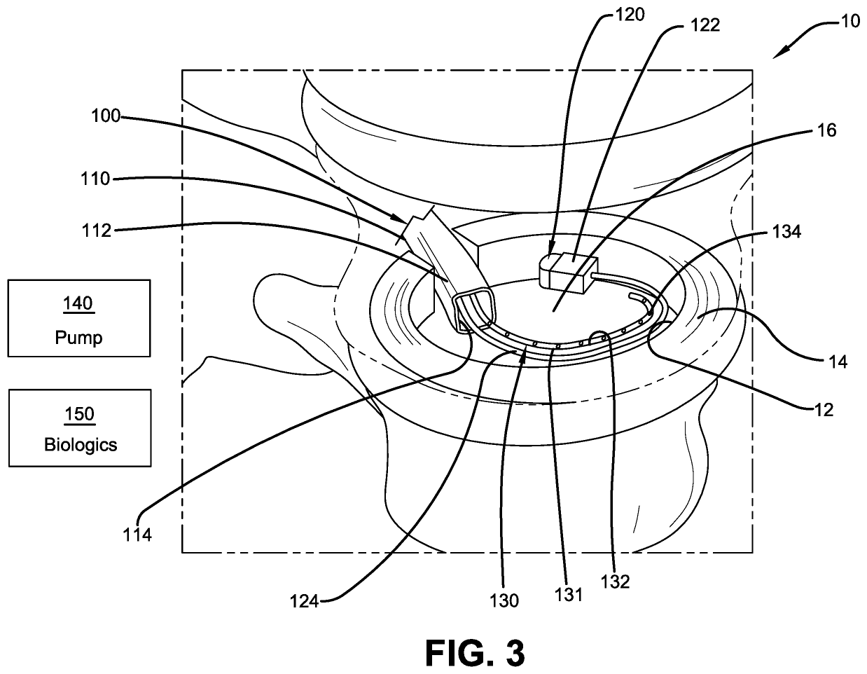

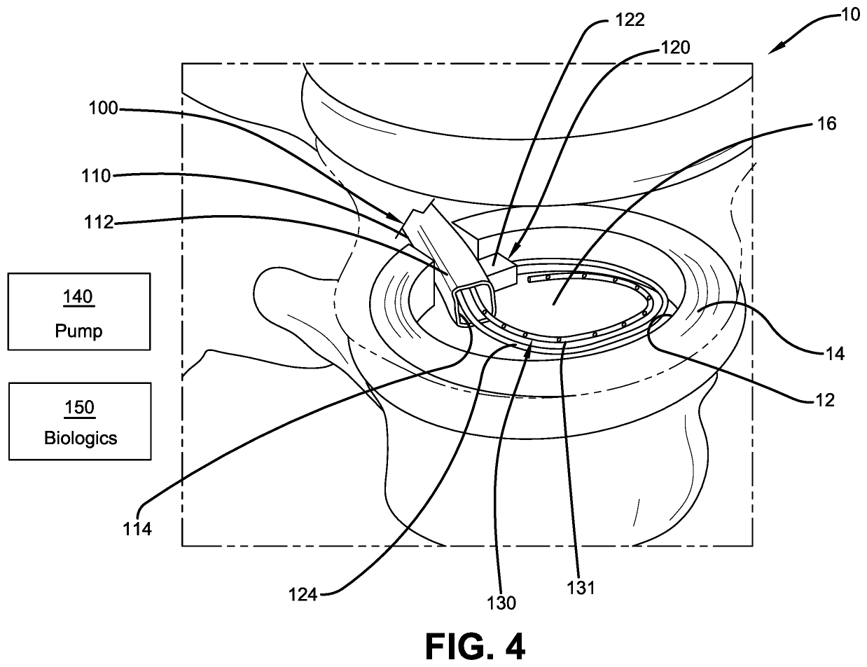

Delivery inserter or probe 120 is preferably comprised of a head portion 122 attached to an end of a coil 124. During the procedure, and as best shown in FIGS. 2-4, probe 120 is inserted into disc space 12 through the continuous longitudinal opening 114 in trocar body portion 112. As probe 120 is inserted into the disc space 12 and head portion 122 comes into contact with the interior circumference of the generally circular disc annulus 14, probe 120 will begin to coil within disc space 12 as shown in the FIGS. Accordingly, coil 124 is preferably an elongated band comprised of a shape memory metal, such as nitinol, though other flexible, shape memory materials may also be used. As explained more fully below, the coiling of probe 120 and coil 124 within disc space 12 enables the surgeon or other medical professional to deliver both the reduced or negative pressure therapy and the regenerative therapy to a large area of the disc space 12 without the need to remove the entire nucleus pulposus 16.

Once the probe 120 and coil 124 are in place, a catheter 130 may also be inserted into disc space 12 via the continuous longitudinal opening 114 in the trocar body portion 112 and follows (and is adjacent to) the coiled path of the probe 120 and coil 124. Catheter 130 may be any catheter known in the art and is commonly comprised of an elongated body portion 131 with a continuous longitudinal opening 132 therein, and a plurality of fenestrations 134 in said body portion 131 that are in fluid communication with the continuous opening 132. Catheter 130 may be attached to, and in fluid communication with, a length of tube (not shown) that is, in turn, in fluid communication with a pump 140 or other vacuum source. Alternatively, catheter 130 may be directly attached to and in fluid communication with pump 140, or other vacuum source without the need for intermediate tubing.

Pump or vacuum source 140 is preferably positioned outside of the patient during the provision of the therapy, and may be any type of pump or vacuum source that is known in the art for creating a reduced or negative pressure environment around a wound or damaged bone. More specifically, pump 140 creates a reduced or negative pressure environment within disc space 12 by pulling air from disc space 12 via the continuous opening 132 and fenestrations 134 in catheter 130. Further, because the catheter 130 is coiled within, and consumes much of, the disc space 12, a greater area of disc space 12 is capable of receiving the reduced or negative pressure therapy than would otherwise be possible.

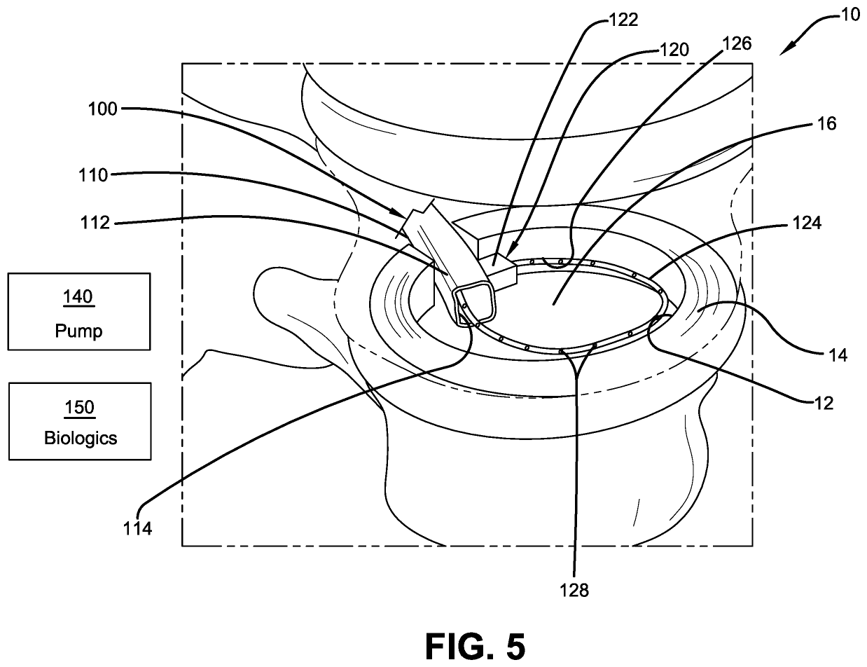

In an alternative embodiment of the present invention, and as best shown in FIG. 5, the cannulated and fenestrated catheter 130 could be eliminated so that the dual therapy device 100 would simply be comprised of the cannulated trocar 110, a cannulated and fenestrated probe or delivery inserter 120, and the pump 140. In this embodiment, the coil 124 of probe 120 would further comprise a continuous longitudinal cannulation or opening 126 therein and a plurality of fenestrations 128 in fluid communication with said continuous opening 126. Further, the probe 120 would be in fluid communication with pump 140, either directly or via a length of hose (not shown) as described above, for creating an on demand reduced or negative pressure environment within disc space 12.

Notwithstanding any of the forgoing, one of ordinary skill in the art will appreciate that the shape and size of the various components of device 100, as shown in FIGS. 1-5, are for illustrative purposes only, and that many other shapes and sizes of said components are well within the scope of the present disclosure. Although the particular dimensions of the device 100 (i.e., length, width, and height) are important design parameters for good performance, device 100 and its various components may be any shape or size that ensures optimal performance during use and is within the overall objective of the present invention.

Having described a preferred embodiment of the dual therapy device of the present invention, a preferred method of its use will now be described in general terms. A medical professional (not shown) desiring to provide reduced or negative pressure therapy and/or regenerative therapy to a portion of a patient's body, such as the disc space 12 of a degenerative or collapsed spinal disc 10, would first sedate the patient and then access the disc space 12 by inserting the trocar 110 (or a similar type device) through the disc annulus 14 and into the disc space 12 under fluoroscopy. Upon insertion, the medical professional would then insert probe 120 into disc space 12 via the continuous longitudinal opening 114 in trocar body portion 112. As probe 120 is inserted into disc space 12 and head portion 122 comes into contact with the inner circumference of the generally circular disc annulus 14, probe 120 and coil 124 will begin to coil around itself within disc space 12, as best shown in the FIGS. 2-5.

Once the probe 120 and coil 124 are in place in disc space 12, the medical professional may insert catheter 130 into disc space 12 via the continuous longitudinal opening 114 in trocar body portion 112. As its inserted, catheter 130 will follow the coiled path of the probe 120 and coil 124, and also coil itself adjacent to probe 120 and coil 124. Once in place, catheter 130 may be attached to, and in fluid communication with, a length of tube (not shown) that is, in turn, in fluid communication with a pump 140 or other vacuum source. Alternatively, catheter 130 may be directly attached to and in fluid communication with pump 140, or other vacuum source. Pump 140 is preferably positioned outside of the patient during the procedure, and may be any type of pump that is known in the art for creating a reduced or negative pressure environment around a wound or damaged bone. More specifically, pump 140 pulls air from the degenerated or collapsed disc space 12 through the fenestrations 134 in the exterior surface of catheter 130 via the longitudinal continuous opening 132 therein. In this manner, reduced or negative pressure therapy may be provided to the entire or substantially entire disc space 12 without the need to remove the entire nucleus pulposus 16 to promote healing and bone growth.

Once the reduced or negative pressure treatment is complete, the catheter 130 may be slowly withdrawn from the disc space 12. As the catheter 130 is being withdrawn, platelet-rich plasma (PRP), stem cells and/or other forms of regenerative biologics 150 may be delivered via the catheter 130, and its continuous opening 132 and plurality of fenestrations 134 therein, and into the void left in the disc space 12 to facilitate healing and bone growth. More specifically, the biological and/or bio-reactive material 150 may be any material currently used to promote healing and bone growth in a disc space 12, such as stem cells, PRPs and the like. Accordingly, the device 100 and method of the present invention, in each of its various possible embodiments, provides a safe, efficient and cost effective manner of delivering both reduced pressure and regenerative therapies to a degenerated vertebral disc in a safe and effective manner.

If the alternative embodiment of the device 100 of the present invention is deployed, the above described method would be very similar except that the cannulated and fenestrated catheter 130 would be eliminated and the dual therapies would be delivered via the continuous longitudinal opening 126 and the fenestrations 128 in probe 120 with the assistance of pump 140. More specifically, a medical professional (not shown) desiring to provide both reduced or negative pressure therapy and regenerative therapy to a portion of a patient's body, such as the disc space 12 of a degenerative or collapsed spinal disc 10, would first sedate the patient and then access the disc space 12 by inserting the trocar 110 (or a similar type device) through the disc annulus 14 and into the disc space 12 under fluoroscopy. Upon insertion, the medical professional would then insert probe 120 into disc space 12 via the continuous longitudinal opening 114 in trocar body portion 112. In the alternative embodiment of the device 100 (i.e., the embodiment without a catheter shown in FIG. 5), probe 120 would be further comprised of a continuous longitudinal opening 126 therein and a plurality of fenestrations 128 in fluid communication with said opening 126. As the cannulated and fenestrated probe 120 is inserted into disc space 12 and head portion 122 comes into contact with disc annulus 14, probe 120 and coil 124 will begin to coil around itself within disc space 12.

Once the probe 120 and coil 124 are in place in disc space 12, probe 120 may be attached to, and in fluid communication with, a length of tube (not shown) that is, in turn, in fluid communication with a pump 140 or other vacuum source. Alternatively, probe 120 may be directly attached to and in fluid communication with pump 140, or other vacuum source. Pump 140 is preferably positioned outside of the patient during the procedure, and may be any type of pump that is known in the art for creating a reduced or negative pressure environment around a wound or damaged bone. More specifically, pump 140 pulls air from the degenerated or collapsed disc space 12 through the fenestrations 128 in the exterior surface of probe 120 via the longitudinal continuous opening 126 therein. In this manner, reduced or negative pressure therapy may be provided to the entire or substantially entire disc space 12 without the need to remove the entire nucleus pulposus 16 to promote healing and bone growth.

Once the reduced or negative pressure treatment is complete, the probe 120 may be slowly withdrawn from the disc space 12. As the cannulated and fenestrated probe 120 is being withdrawn, PRP, stem cells and/or other forms of regenerative biologics 150 may be delivered via the probe 120, and its continuous opening 126 and plurality of fenestrations 128 therein, and into the void left in the disc space 12 to facilitate healing and bone growth. More specifically, the biological and/or bio-reactive material 150 may be any material currently used to promote healing and bone growth in a disc space 12, such as stem cells, PRPs and the like. Accordingly, the alternative device and method of the present invention provides a safe, efficient and cost effective manner of delivering both reduced pressure and regenerative therapies to a degenerated vertebral disc in a safe and effective manner.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A stem cell therapy system for injecting a biological material into a patient's disc space comprising:
a trocar device;
a probe; and
a catheter.

2. The system of claim 1, wherein said trocar device is cannulated, and wherein said catheter is cannulated and fenestrated.

3. The system of claim 1, wherein said catheter is in fluid communication with a vacuum source.

4. The system of claim 1, wherein the probe is coiled within the disc space.

5. The system of claim 1, wherein the catheter is coiled within the disc space.

6. The system of claim 1, wherein the probe is comprised of a shape memory material.

7. The system of claim 1, wherein the catheter delivers biological material to the disc space.

8. The system of claim 3, wherein the vacuum source creates a reduced pressure environment in the disc space.

9. The system of claim 1, wherein the probe and the catheter are inter-coiled in the disc space.

10. A system for providing both reduced pressure therapy and regenerative therapy to a vertebral disc space comprising:
a cannulated device for accessing the disc space;
a probe; and
a vacuum source.

11. The system of claim 10, wherein the probe extends through the cannulated device and into the disc space where it is partially coiled therein.

12. The system of claim 10, wherein the probe is further comprised of a continuous longitudinal opening therein.

13. The system of claim 12, wherein the probe is further comprised of a plurality of fenestrations that are in fluid communication with said continuous longitudinal opening.

14. The system of claim 10, wherein the probe is comprised of a head portion and a coil, and further wherein the coil is comprised of a shape memory material.

15. The system of claim 14, wherein the shape memory material is comprised of nitinol.

16. The system of claim 10, wherein the probe is in fluid communication with said vacuum source.

17. A method of providing therapy to a disc space of a patient comprising the steps of:
accessing the disc space with a cannulated trocar;
inserting a probe into the disc space via the cannulated trocar, wherein the probe is coiled within the disc space; and
creating a negative pressure environment within the disc space.

18. The method of claim 17 further comprising the step of inserting a catheter into the disc space before creating the negative pressure environment within the disc space.

19. The method of claim 17 further comprising the step of injecting a biological material into the disc space.

20. The method of claim 19, wherein the biological material is injected as the probe is being removed from the disc space.
